(19) United States
(12) Patent Application Publication
(10) Pub. No.: US 2013/0184781 A1
(43) Pub. Date: Jul. 18, 2013

(54) Brain Stimulation for Enhancement of Learning, Motivation, and Memory

(71) Applicants: Emad N. Eskandar, Nahant, MA (US); Shaun Rajni Patel, Chelsea, MA (US); John Gale, Boston, MA (US)

(72) Inventors: Emad N. Eskandar, Nahant, MA (US); Shaun Rajni Patel, Chelsea, MA (US); John Gale, Boston, MA (US)

(21) Appl. No.: 13/808,331

(22) PCT Filed: Jul. 6, 2011
(86) PCT No.: PCT/US11/43025

§ 371 Date: Mar. 20, 2013

Related U.S. Application Data
(60) Provisional application No. 61/361,761, filed on Jul. 6, 2010.
(60) Provisional application No. 61/361,779, filed on Jul. 6, 2010.
(60) Provisional application No. 61/361,770, filed on Jul. 6, 2010.

(51) Int. Cl. A61N 1/36

(52) U.S. Cl. CPC A61N 1/36082
USPC 607/45

(57) ABSTRACT

A system and method for stimulating a brain structure of a patient for treating a condition. A stimulating electrode is inserted into a brain structure of the patient. After inserting the stimulating electrode into a brain structure of the patient, the patient is prompted with a task. When the task is completed by the patient, the stimulating electrode is used to transmit a stimulating signal into the brain structure of the patient. In an alternative implementation, the patient's brain is monitored for the existence of a particular condition. When the condition is detected, the stimulating electrode is used to transmit a stimulating signal into the brain structure of the patient.

### CROSS REFERENCE TO RELATED APPLICATIONS

This application is based on, claims the benefit of, and incorporates herein by reference U.S. Provisional Application Ser. No. 61/361,761; filed Jul. 6, 2010, and entitled “A Brain Stimulation System for Enhancement of Memory,” U.S. Provisional Application Ser. No. 61/361,779, filed Jul. 6, 2010, and entitled “Method for Increasing Dopamine Release in the Striatum and Other Brain Areas using Electrical Stimulation,” and U.S. Provisional Application Ser. No. 61/361,770, filed Jul. 6, 2010, and entitled “Two Streams of Information Processing in the Anterior Striatum.”

### FIELD OF THE INVENTION

The present invention relates to systems and methods for enhancing memory and association formation and, more particularly, to a system and method for using electrical stimulation of brain structures to enhance memory and association formation within a subject.

### BACKGROUND OF THE INVENTION

Brain disorders or injuries such as stroke, traumatic injury, Alzheimer's disease (AD), dementia and autism represent major public health concerns. In regard to stroke, the CDC estimates a nationwide prevalence of 2.6% or about 5,839,000 patients. The estimated cost of treatment is $62.7 billion in 2007. Traumatic brain injury also affects a large number of patients in the USA, estimated by the CDC to be about 1,400,000 patients per year. There is currently tremendous public concern about autism. The most recent data from the CDC suggests a prevalence of about 6.7 per 1000 children, or about 163,000 patients nationwide. Unfortunately, the treatment for all three disorders is largely supportive. In the case of stroke or temporary brain injury, the goal is to weather the acute stage and then undergo an extended period of rehabilitation. In the case of autism, the treatment is largely based on behavior modification approaches. In none of these disorders is there a rational treatment aimed at utilizing the remaining brain circuitry to accelerate the learning process.

Deep brain stimulation (DBS) can be used to treat various neurological disorders such as movement disorders (e.g., Parkinson Disease and Essential Tremor). In spite of the number of applications for DBS, currently available technology for commercial DBS systems is primitive and inflexible. In general, existing DBS systems are often based on cardiac pacemakers and have not fundamentally changed for over two decades in spite of the enormous strides in electrical engineering technology and other fields. These DBS systems are generally limited to operating on a small number of channels and typically operate in an “open-loop” fashion, where stimulation is delivered to the patient's brain continuously or according to a pre-determined algorithm regardless of the patient's current status or progress. As such, these conventional DBS systems are unable to monitor the status or progress of a patient in order to deliver therapeutic stimulation that is more tailored to that particular patient's needs. The devices have no extrinsic or intrinsic feedback to control the devices to provide optimum care for a patient. Moreover, current systems cannot be used to treat traumatic brain injury, stroke, AD, autism, and many other disorders.

### SUMMARY OF THE INVENTION

The present invention relates to systems and methods for enhancing learning, motivation, and/or memory formation and, more particularly, to a system and method for using electrical stimulation of brain structures to enhance memory and learning within a subject.

In one implementation, the present invention is a method for stimulating at least one of memory formation and association formation in a patient. The method includes inserting a stimulating electrode into a brain structure of the patient, and prompting the patient with a task. When the task is completed by the patient, the method includes using the stimulating electrode to transmit a stimulating signal into the brain structure of the patient.

In another implementation, the present invention is a method for stimulating at least one of memory formation and association formation in a patient. The method includes inserting a stimulating electrode into a brain structure of the patient, and monitoring electrical activity in the brain of the patient for theta oscillations. When theta oscillations are detected in the electrical activity of the brain of the patient, the method includes using the stimulating electrode to transmit a stimulating signal into the brain structure of the patient.

In another implementation, the invention is a system for stimulating a brain structure of a patient for treating a condition. The system includes a stimulating electrode configured to be disposed into a brain structure of the patient, and a controller. The controller is configured to prompt the patient with a task, and, when the task is completed by the patient, use the stimulating electrode to transmit a stimulating signal into the brain structure of the patient.

In another implementation, the present invention is a system for stimulating at least one of memory formation and association formation in a patient. The system includes a stimulating electrode configured to be disposed into a brain structure of the patient. The system includes a controller configured to monitor electrical activity in the brain of the patient for theta oscillations, and, when theta oscillations are detected in the electrical activity of the brain of the patient, use the stimulating electrode to transmit a stimulating signal into the brain structure of the patient.

The foregoing and other aspects and advantages of the invention will appear from the following description. In the description, reference is made to the accompanying drawings which form a part hereof, and in which there is shown by way of illustration at least one embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is an illustration of a coronal section of the brain showing the region containing the hippocampus, nucleus basalis, and mammillary bodies.

FIG. 2 is a coronal section of a brain showing the caudate nucleus and nucleus accumbens.

FIG. 3A is an illustration of an embodiment of a system for implementing therapeutic high-frequency stimulation.

FIG. 3B is a block diagram illustrating some of the functional components of the present system for implementing therapeutic high-frequency stimulation.

FIG. 4A is a flowchart illustrating a method for closed-loop monitoring and delivery of a stimulation signal to a patient that can be used for treating memory or brain disorders.

FIG. 4B is a flowchart showing an alternative method for delivering a stimulating signal to a patient for delivery of a stimulation signal to a patient that can be used for treating memory or brain disorders.

FIG. 5 illustrates functional components of an implantable device that may be used in accordance with the present disclosure (see, for example, element 125 of FIG. 3).

FIG. 6 is a block diagram illustrating functional components of the present implantable device (e.g., device 125 of FIG. 3A).

FIG. 7 is an illustration showing an implantable device of the present system implanted in a patient's head.

FIG. 8A is an illustration of stimulating and recording electrodes disposed within a subject's caudate in accordance with the present disclosure.

FIGS. 8B-8D are graphs illustrating changes in dopamine oxidation in different regions within the subject's brain.

FIG. 9A is an illustration of a path of a stimulating electrode within a subject's midbrain.

FIGS. 9B-9D are graphs illustrating changes in dopamine oxidation in different regions within the subject's brain.

### DETAILED DESCRIPTION

The present invention relates to systems and methods for enhancing memory and association formation and, more particularly, to a system and method for using electrical stimulation of brain structures to enhance memory and association formation within a subject.

Recent studies have demonstrated that intermittent electrical stimulation of various brain structures including the caudate, a nucleus in the basal ganglia, can significantly enhance the rate of visual-motor learning in primates (Williams Z M and Eskandar E N (2006). Selective enhancement of associative learning by microstimulation of the anterior caudate. Nat Neurosci. 6 April; Vol. 9, No. 4, pp. 562-568). There is also additional data suggesting that the effects of stimulation are mediated by phasically enhancing dopamine release. In essence, stimulation appears to function in a manner analogous to spontaneous learning, but in an amplified fashion that is under experimental control.

It is commonly held that the learning rate of different animals or humans is relatively fixed. However, this experimental data suggest that it is possible to enhance learning beyond baseline rates, suggesting that there is in fact considerable room for improvement. In other words, even though primates or humans may have a natural rate of learning new associations or generating new memories, the underlying circuitry maybe able to learn at much higher rates. Thus, the present system and method utilizes electrical stimulation of various brain structures to directly and specifically enhance associative learning thereby speeding the process of recovery following brain injury or in the case of dementing disorders.

There is considerable evidence that there is some degree of plasticity following brain injury. The typical finding is that the area surrounding, for example, a discrete infarct can to some extent subsume the function of the injured area. However, this process is slow and far from perfect. In addition, it is not easily accessible. The only way to engage this process is through a long and tedious course of rehabilitation involving considerable repetition. Even then, the results are mixed and difficult to predict. A major issue is the lack of information optimizing this process.

The present system and method, therefore, uses direct brain stimulation to enhance the acquisition of certain capabilities (e.g. task learning), remedy brain injuries and/or dementing disorders, and/or modify behavior (e.g., by diminishing the effects of depression or motivational problems). A stimulating device is first implanted into the patient's brain, for example in the caudate, nucleus accumbens, hippocampus, striatum, nucleus basalis, mammillary bodies, subthalamic nucleus or midbrain (these areas have dopaminergic terminals, projections, or cell bodies). In one implementation, the stimulating device is configured to communicate with an external piece of equipment. The patient is situated in an environment where a learning task may be administered (for example at home or in a clinic). The learning task is automated such that when the patient responds appropriately to the given task the external equipment automatically commands the implanted device to stimulate the targeted area of the patient's brain. In an alternative embodiment, the stimulating device may be configured to detect certain conditions within the patient's brain and then trigger stimulation when those conditions are detected. One example condition is the presence of theta oscillations occurring with the patient's hippocampus. Other conditions include the presence of alpha, beta, or gamma oscillations in various structures within the patient's brain, detection of a particular or single neuron firing, or the presence of particular levels of neurotransmitters such as dopamine, glutamate, or serotonin. As described below, particular oscillations occurring within the patient's brain (or, in fact, any of these conditions) can indicate that learning is actively occurring with the patient's brain. After detecting the condition, the stimulating device delivers a stimulating signal to the target area of the patient's brain.

In either implementation, by providing the stimulation during a controlled window of time (e.g., within a window occurring shortly after the patient successfully completes a task or a particular brain condition is detected), the patient's ability to learn and perform the task is enhanced. This enhanced learning is useful in a number of circumstances including various brain injuries or conditions. The present system may be used, for example, during recovery of motor skills or speech. It may be supervised in a semi-automated fashion by a clinician or by a family member, or may be completely automated.

The present system (also referred to herein as the neurological rehabilitation system (NRS)), therefore, can be used to rehabilitate a wide range of brain disorders, by harnessing and augmenting the brain's innate memory circuitry. The present system may be implemented via an implant that could be completely contained within the head of a patient and accessible via a wireless communications channel. The present system may be triggered and recharged by wireless telemetry using a specialized cap that contains an external unit. The external unit is configured to communicate wirelessly with an implanted subcutaneous stimulator unit and, optionally, with a computer.

In the present system, an external device (e.g., a computer in communication with the stimulating device) may be configured to execute software that is configured to rehabilitate patients on a broad range of tasks. For example, the computer may cause the present system to enhance object recognition memory. In that case, the present system delivers stimulation based on the patient accurately recalling the name of an object or the object that corresponds to a particular name. The program can be adaptive, beginning with simple objects and gradually progressing to more complex or subtle object target size as accuracy improves. In one implementation, the computer plays pre-recorded words followed by a selection of visual images to enhance speech recognition. In that case, the patient selecting the correct corresponding image would then be coupled with appropriate stimulation. It is possible to enhance simple cognitive tasks such as basic mathematics by presenting simple problems and reinforcing selection of correct answers or one can use other simple decision-making tasks. Conversely, it may be possible to attenuate severe anxiety or depression by reinforcing the provocative stimuli with a rewarding stimulus.

By stimulating particular regions of the brain, either in response to external cues (e.g., observations made by a treating physician, or responses detected by a computer), internal cues (e.g., monitoring of particular brain conditions such as oscillations or neurotransmitter levels, as described above), or continuously, a number of brain disorders can be treated. The delivery of stimulating signals in accordance with this disclosure serves to facilitate the creation of new memories or associations that can be lost as the result of the patient suffering from a particular disease, condition, or trauma.

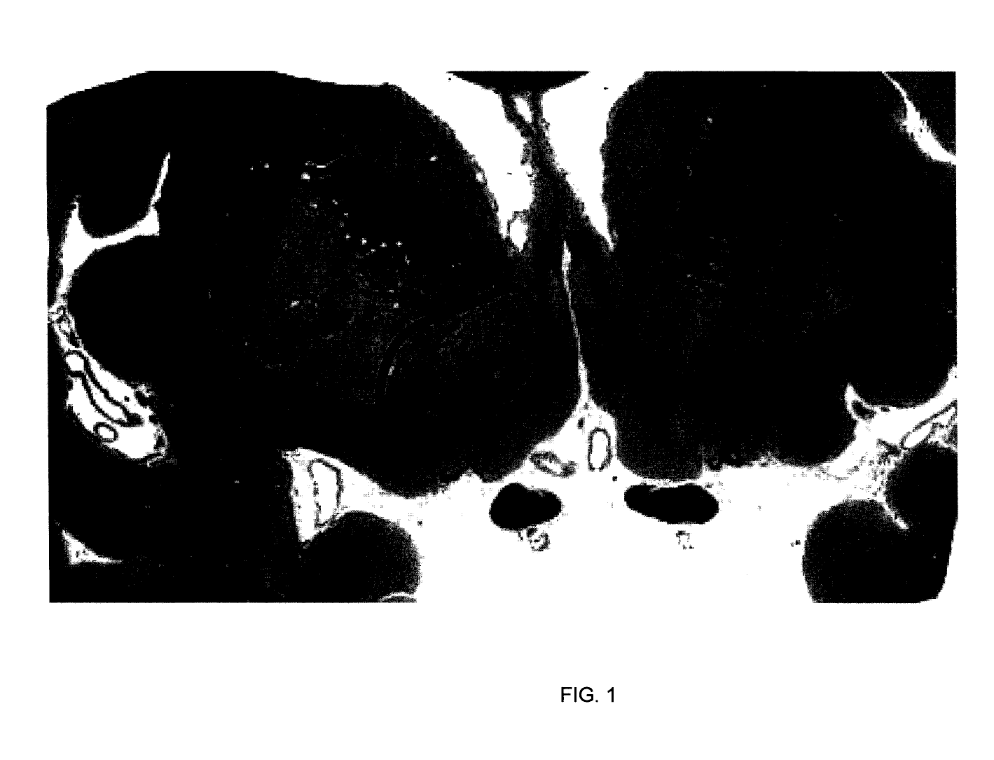

In the present system, therefore, appropriately-timed high-frequency stimulation in the caudate, nucleus accumbens, hippocampus, nucleus basalis, and/or mammillary bodies can enhance memory formation and retention, treat brain injuries, or mitigate conditions such as depression or motivational issues. As an example, FIG. 1 is an illustration of a coronal section of the brain showing the region containing the hippocampus, nucleus basalis, and mammillary bodies. Treatment within those regions may be of great use in treating patients with stroke, traumatic brain injury, memory disorders such as AD, or other conditions. The stimulation may occur at several different areas using intermittent stimulation triggered by the subject's performance on appropriate tasks.

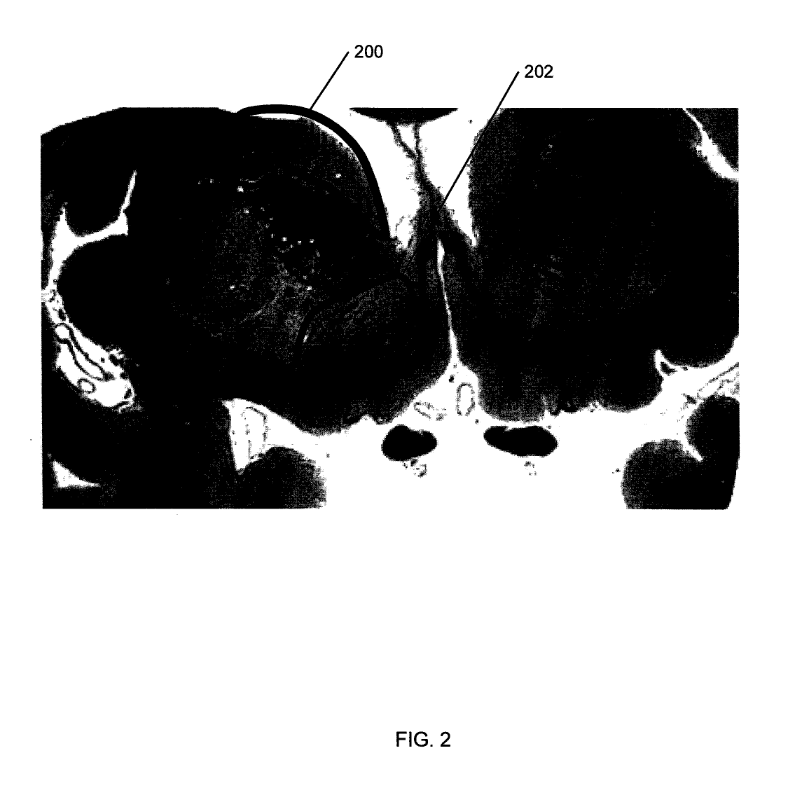

Other brain structures that are involved in memory formation are found within the corpus striatum. The caudate nucleus (Cd) in the dorsal striatum receives input from the dorsal lateral prefrontal (DLPFC) and parietal cortices and from midbrain dopaminergic neurons. The anterior Cd is involved in the acquisition of new motor-limb behavior, with dopamine playing a role in this function. The Cd projects to the dorsal globus pallidus internus which projects to the thalamus, which in turn, ultimately projects back to the cortex. The prefrontal cortex is involved in associating particular sensory stimuli with specific motor behaviors. In relative contrast, the nucleus accumbens (NAc) in the ventral striatum occupies a unique and central position due to the complexity and richness of its connections. The NAc receives inputs from the orbitofrontal cortex, hippocampus, amygdala, from dopaminergic neurons in the midbrain, and from serotonergic neurons in the raphe nucleus. FIG. 2 is a coronal section of a brain showing Cd 200 and NAc 202.

Each of these inputs is associated with different types of information. Orbitofrontal inputs carry information about the relative value of different stimuli. The basolateral amygdala conveys information about the emotional valence of stimuli, particularly those that evoke strong emotions such as fear. The hippocampus is associated with visual-spatial memory. Finally, dopaminergic neurons signal both the novelty of stimuli and the difference between expected and actual reward. Along with its primary projection to the dorsomedial thalamus, which projects back to the orbitofrontal cortex, there is a strong projection to dopaminergic areas of the midbrain. Thus, the NAc is in a position to regulate the level of dopamine that is released to other areas. The NAc occupies a central position linking powerful drives with different behaviors.

The anterior striatum is characterized by a strong input from midbrain dopaminergic neurons. The dopaminergic neurons have very characteristic properties. They fire most robustly to unexpected reward, moderately to expected reward, and have a depression in their otherwise tonic baseline in response to an expected but omitted reward. These neurons have been viewed as conveying a signal regarding an error in the temporal prediction of reward. Based on these findings, the striatum is considered important in reinforcement learning. In reinforcement learning the system or network (the actor) is given evaluative feedback based on the correctness of a given response (the critic). The actor has been construed as the striatum, while the critic is construed as the dopaminergic neurons. The feedback that the critic provides to the actor consists of the reward prediction error (RPE)—the difference between expected reward and actual reward. Thus, if the consequences of a response to a stimulus are better than expected (RPE>0), or worse then expected (RPE<0) then the response should be strengthened or weakened respectively. If the consequences are as expected, (i.e., RPE=0) the response tendency should remain unchanged. Importantly, phasic dopamine release has been shown to result in long lasting potentiation of active cortical-striatal synapses or circuits.

Caudate activity is closely correlated with the rate of learning, and peaks during the steepest portion of the learning curve (i.e., when new visual stimuli and limb-movement associations are being formed. There is a similar modulation of caudate activity during an ocular-motor learning task. This demonstrates that the caudate is involved in learning across multiple modalities, for example both arm and eye movements, reflecting the caudate's broad connections with frontal and parietal cortices. Moreover, this behavior suggests that the caudate is involved in the executive aspects of rapid associative learning.

The caudate is not active at the very beginning of a block of trials (when the subject is ‘naïve). The caudate is also less active, when the subject has mastered a particular association. At that point the association has become habitual, and no new learning is required to establish the association. Instead, the caudate is most active during the steep part of the learning curve when new associations are being made. It is precisely this period of time when applied stimulation to caudate is most effective in enhancing learning. In contrast to existing DBS systems which provide only continuous stimulation, the present system, therefore, delivers a stimulating signal for a defined time period (e.g., 1 sec, at 200 Hz, 200 μA, 0.1 ms/phase square wave) during the feedback period to be most effective.

In some cases, improved learning or memory formation is associated with increased levels of dopamine in various brain structures, such as the anterior caudate nucleus, as described above.

Experimentation has determined that high-frequency stimulation of brain structures results in significantly more dopamine release than low-frequency stimulation. Accordingly, high-frequency stimulating signals, when applied to the brain, can transiently increase dopamine concentration in, for example, the caudate resulting in enhanced learning-related striatal plasticity. In addition, experimentation has shown that electrical stimulation operates differentially on specific neuronal elements; where the stimulation parameters for maximal dopamine release are different for the caudate, putamen and medial-forebrain bundle (MFB). These findings suggest that intermittent striatal stimulation can be used to enhance visual-motor learning after various kinds of brain injury.

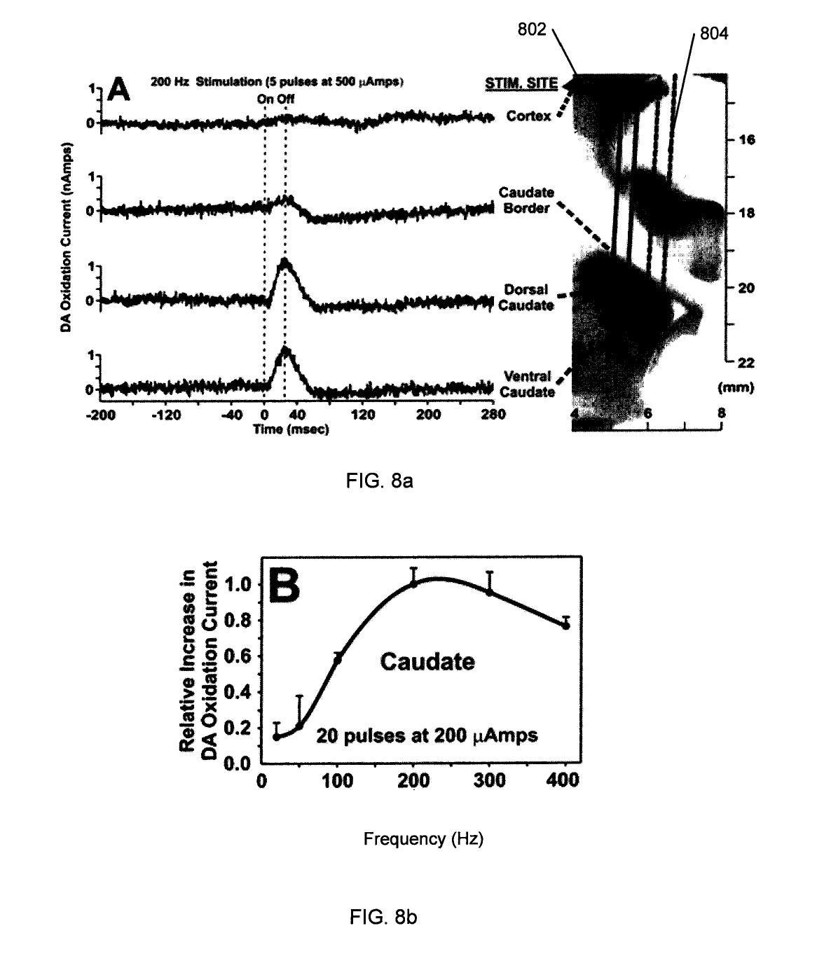

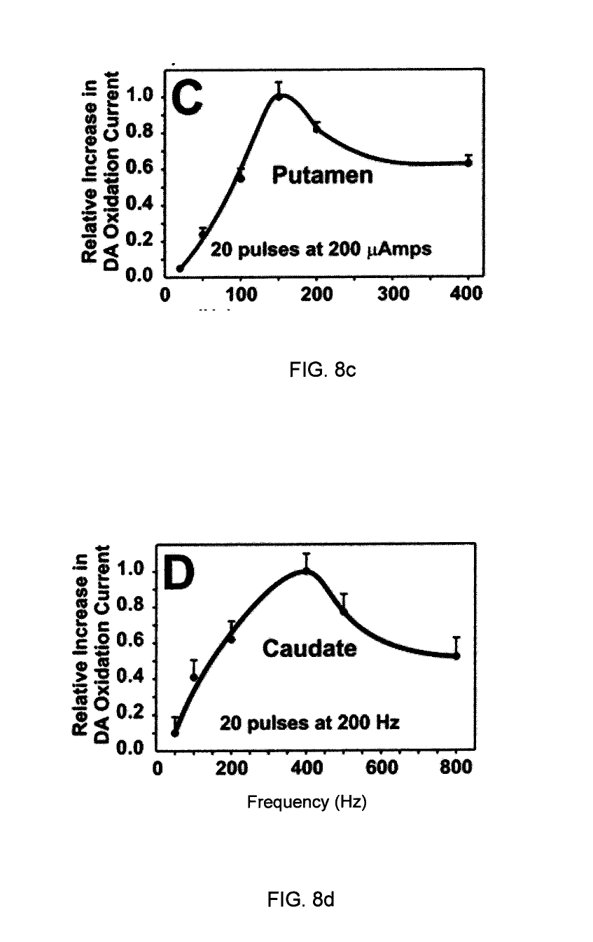

In one such experiment, stimulating electrodes were introduced into the caudate of an adult monkey. FIG. 8B shows a representation of a portion of the caudate of a monkey into which has been placed stimulating electrodes 802 (shown by the solid gray line) and recording electrode 804 (shown by the dashed gray line). The recording electrode is configured to detect dopamine levels proximate to the recording electrode. In one implementation, the recording electrode uses amperometry to measure dopamine levels.

Fixed potential amperometry is an electrochemical recording procedure which has been shown to provide a reliable in vivo method to monitor the processes of dopamine release and reuptake in dopaminergic terminal sites in the brain (C. D. Blaha and A. G. Phillips, “A critical assessment of electrochemical procedures applied to the measurement of dopamine and its metabolites during drug-induced and species-typical behaviours,” Behav Pharmacol, vol. 7, pp. 675-708, November 1996. M. F. Suaud-Chagny, “In vivo monitoring of dopamine overflow in the central nervous system by amperometric techniques combined with carbon fibre electrodes,” Methods, vol. 33, pp. 322-9, August 2004). This method also offers the best temporal monitoring resolution of all in vivo electrochemical methods to date (>10K samples/sec). Carbon-fiber electrodes, in combination with fixed potential amperometry, permit quantitative detection of dopamine overflow evoked by electrical-stimulation of dopaminergic fibers in the MFB or in the striatum. More recently, amperometry has been used to monitor forebrain dopamine release in response to natural stimuli, such as light pulses in rats. Collectively, these studies have confirmed the utility of amperometric procedures in measuring electrically-evoked dopamine release. With regards to chemical specificity, several studies have shown unequivocally that no other oxidizable compound (e.g., DOPAC or ascorbate), except dopamine, is detected at the tip of carbon microelectrodes in vivo when using stimulations of durations<50 sec. Adenosine is also electroactive and can thus be measured with fixed potential amperometry using carbon electrodes, however it oxidizes at a much higher potential (+1.3 V) than dopamine (+0.6 V) (see M. L. Huffman and B. J. Venton, “Carbon-fiber microelectrodes for in vivo applications,” Analyst, vol. 134, pp. 18-24, January 2009). In addition, unlike fast-scan cyclic voltammetric recordings, the amperometric signal is not prone to temporal distortion by changes in pH or by analyte adsorption to the electrode surface.

In one experiment, the recording electrode included an active carbon surface (mean 0.83 mm2) produced by turning one end of a carbon rod on an ultrafine sanding disk, creating an exposed conical tip. A guide tube was then temporarily introduced through the grid at the specific target hole to perforate the dura prior to introducing the carbon-fiber recording electrode. A stainless-steel/Ag/AgCl auxiliary/reference electrode combination was placed in contact with the surface of the ipsilateral cortex. A custom-made Faraday cage was then used to reduce extraneous noise. Once the recording and auxiliary/reference electrodes were in place, a fixed positive potential (+0.8 V) was applied to the recording electrode and the oxidation current was monitored continuously (10 KHz/s) with an electrometer (e-corder/picostat system, eDAQ Incorporated, CO, USA). The data was notch and low-pass filtered at 60 Hz and 10 Hz, respectively, and recorded online. All experiments were performed a minimum of 30 min after the implantation of the recording electrodes in order to allow the amperometric signal to reach a steady-state baseline response. Electrical stimulations consisting of positive monophasic 500 μsec duration pulses at different frequencies and current intensities (Iso-Flex/Master-8; AMPI, Jerusalem, Israel), described for each experiment below, were applied no more frequently than every 30 sec during each experiment.

As shown in FIG. 8A, the stimulating electrodes were advanced along a parallel trajectory to the recording electrode from the level of the cortex to the caudate. As shown in FIG. 8A (left panel), electrical-stimulation was applied at different sites along the trajectory of the electrodes while caudate dopamine release in terms of oxidation current was recorded by the carbon-fiber recording electrode 804.

High-frequency electrical-stimulation in the dorsal and ventral caudate evoked a significant increase in local dopamine extracellular concentrations as evidenced by a stimulus time-locked increase in the amperometrically recorded oxidation signal. In contrast, stimulation of the white matter or cortex above the caudate did not evoke a change in the dopamine oxidation current.

To characterize stimulation-evoked dopamine release within the caudate, the stimulation frequency was varied between 20-400 Hz while keeping the other parameters unchanged (i.e., 20 pulses at 200 μAmps). As shown in FIG. 8B, caudate stimulation at 200 and 300 Hz evoked a significantly greater increase in dopamine release compared to higher (400 Hz) and lower (50 and 100 Hz) stimulation frequencies (p<0.001 analysis of variance (ANOVA) for higher and lower frequencies). As shown in FIG. 8C, in the putamen stimulation at 150 Hz evoked a significantly greater increase in local dopamine release compared to higher (200-400 Hz) and lower (20-100 Hz) stimulation frequencies (p<0.001 ANOVA for higher and lower frequencies). In addition to frequency dependence, evoked dopamine release in the caudate was current intensity-dependent. As shown in FIG. 8D, 200 Hz stimulation at 400 μAmps evoked a significantly greater increase in local dopamine release relative to higher and lower stimulation current intensities (500-800 and 50-200 μAmps, respectively; p<0.001 ANOVA for higher and lower currents).

Accordingly, the experiment shows that a 1 sec stimulation at 200 Hz in the caudate evokes a significantly higher increase in local dopamine release compared to a 1 sec stimulation at 20 Hz (200 μAmps; p<0.001, Wilcoxon rank-sum test; e.g., FIGS. 8A and B) in both caudate and putamen. In addition, high-frequency stimulation (200 Hz) evoked a relatively greater increase in dopamine release in the caudate compared to the putamen (p=0.002, Wilcoxon rank-sum test; FIG. 8A).

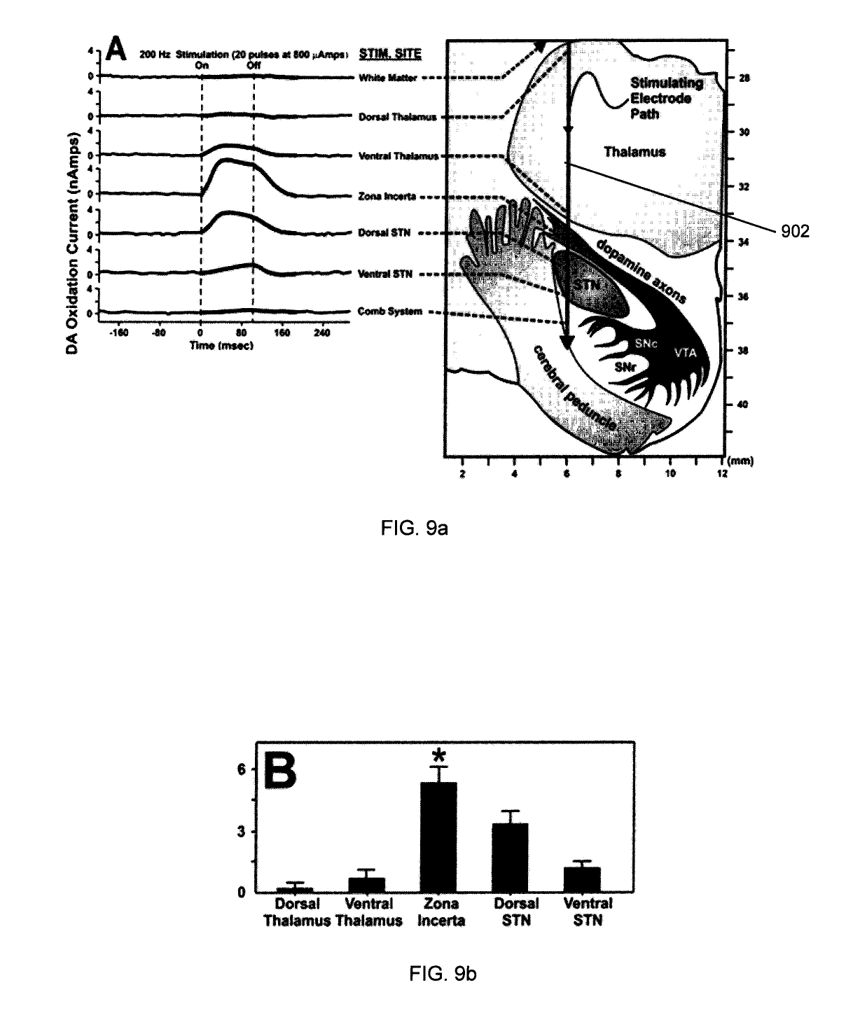

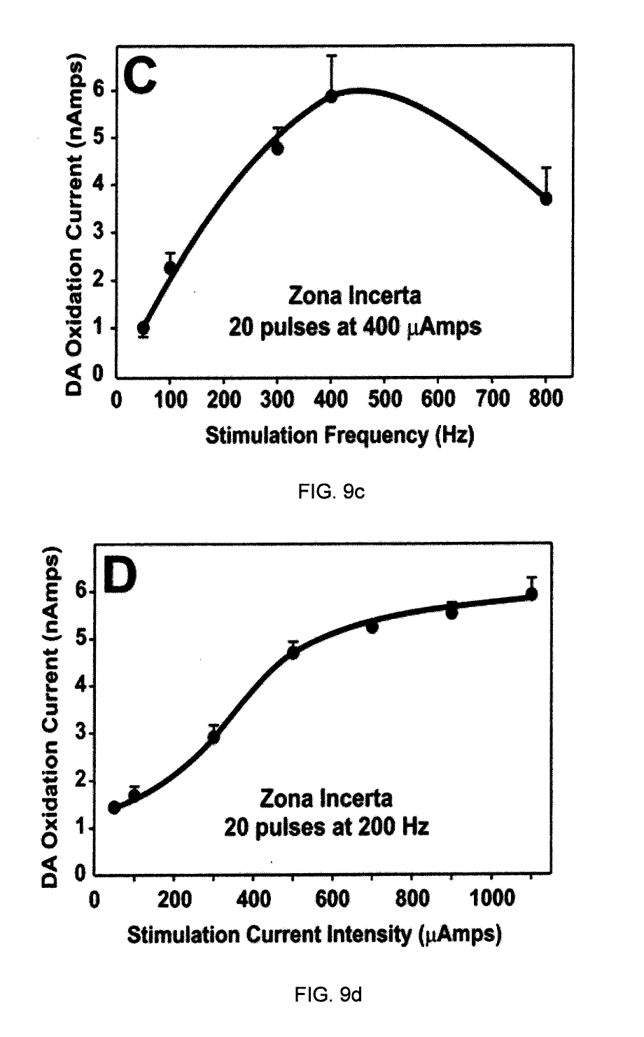

To verify that stimulation-evoked changes in the amperometric signal corresponded specifically to changes in extracellular concentrations of dopamine, a carbon-fiber composite electrode was advanced into the caudate while stimulation was applied at different points along a trajectory beginning in white matter, and traversing the thalamus, MFB, STN, and ending in the comb system. The right panel of FIG. 9A illustrates the path 902 of the stimulating electrode. Moving along path 902, the stimulating electrode traversed ascending dopaminergic axons within the MFB in the region of the zona incerta. Stimulation in the white matter, dorsal thalamus, and comb system (ventral to the STN) did not evoke significant dopamine release within the caudate. In contrast, stimulating the most ventral border of the thalamus, zona incerta, and dorsal and ventral STN evoked significant increases in caudate dopamine release as shown by the left panel of FIG. 9A. Zona incerta/MFB stimulation induced the greatest increase in caudate dopamine release (paired t-test, p=0.01), compared to stimulation of adjacent sites (FIG. 9B).

Like local caudate stimulation, zona incerta/MFB stimulation-evoked dopamine release at 400 μAmps was frequency-dependent as shown by FIG. 9C. Stimulation at 400 Hz evoked a significantly greater increase in caudate dopamine release compared to higher (800 Hz) or lower (50-300 Hz) stimulation frequencies (p=0.02 and p=0.03, respectively, ANOVA). In addition, caudate dopamine release evoked by MFB stimulation (200 Hz) was also current intensity-dependent (FIG. 9D) showing a sigmoidal increase in the evoked dopamine release in the caudate with progressive increases in stimulation current intensity (50-1100 μAmps).

Accordingly, this experiment demonstrates that high-frequency stimulation in the anterior caudate enhances extracellular concentrations of dopamine and, consequently, can thereby enhance learning. Additionally, this experiment shows that the maximal evoked dopamine release is frequency and substrate dependent. In other words, different neuronal substrates (i.e., caudate, putamen and MFB) appear to have particular stimulation parameters that optimally increase dopamine concentrations.

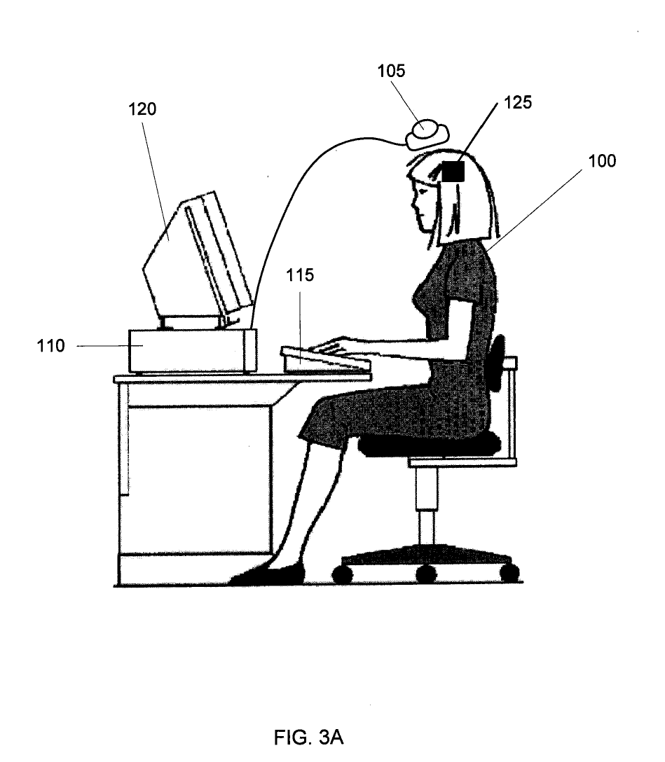

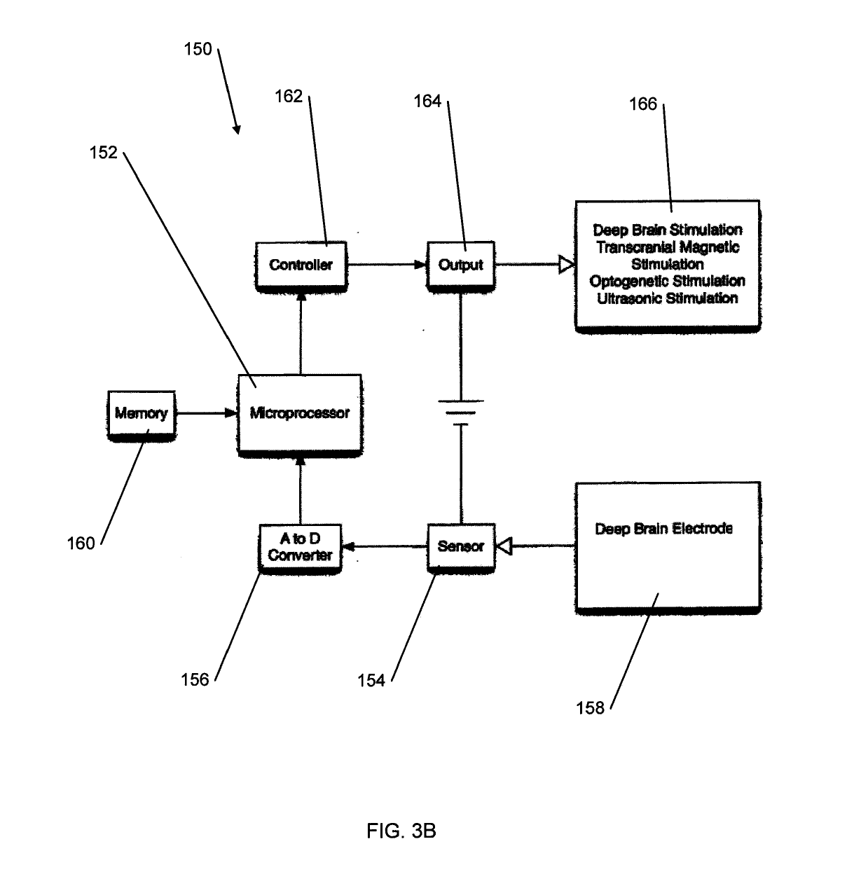

FIG. 3A is an illustration of an embodiment of a system for implementing the therapeutic high-frequency stimulation described above. The system includes an implantable device (represented by block 125) such as an electrode or other stimulator that is implanted into a target brain structure of patient 100, such as the caudate, nucleus accumbens, or hippocampus. The device illustrated in FIG. 3A may optionally include one or more sensors (either integrated into the electrode, or provided by other distinct implantable devices) for monitoring activity within the patient's brain. For example, the implantable device 125 may incorporate one or more recording electrodes for monitoring a condition within the patient's brain or within a particular structure of the patient's brain. Example conditions include alpha, beta, theta, or gamma oscillations within a particular brain structure, single neuronal firing, or detection of neurotransmitters such as dopamine, glutamine, or serotonin. Alternatively, the patient's brain could be monitored using one or more of a scalp electroencephalogram (EEG) or cortical EEG.

Device 125 is configured to deliver therapeutic high-frequency stimulating electrical signals to the region of the brain in which device 125 is implanted. Device 125 may be implanted into, for example, the caudate and nucleus accumbens (e.g., for treatment of brain injury and stroke), or the hippocampus, nucleus basalis and/or mammillary bodies (e.g., for assisting in memory formation). Device 125 is connected to an appropriate stimulator device that may itself be disposed within patient 100, or external to patient 100. In either case, the stimulator controls delivery of the stimulating signals to the brain through device 125. The stimulator may communicate with device 125 through a wired or wireless connection. When the stimulator is external to patient 100 it may be preferable, though not necessary, that the stimulator communicate wirelessly with device 125.

The stimulator of implantable device 125 is configured to wirelessly communicate with an external wand 105 connected to a personal computer (PC) 110. The PC 110 provides a task using a user interface (e.g., the screen, speakers, or other user interface device) for the patient 100 to perform. The task may include, for example, using keyboard 115 or another user interface of PC 110 to track a target on the display screen 120, identify a face, touch a particular region of a touch screen, or identify an object verbally, for example. Generally, the task is selected based upon patient 100's condition. When the patient performs the task successfully, PC 110 commands the implanted device to stimulate the patient's brain to enhance the acquisition of the task. Other motor tasks may include using a computer mouse, or the manipulation of other objects. Task requiring patient ambulation may be prompted using other interface devices besides a keyboard 115.

As described below, device 125, in combination with an appropriate stimulator (which may be incorporated directly into device 125) may operate in a number of modes. For example, device 125 may continuously transmit a stimulating signal into the patient's brain, or may only transmit the signal when instructed to do so by an external system (e.g., PC 110). PC 110, for example, may instruct device 125 to transmit a stimulating signal in response to input provided by a physician monitoring patient 100. Alternatively, PC 110 may instruct device 125 to transmit the stimulating signal after detecting the existence of a particular condition. For example, PC 110 may cause the stimulating signal to be transmitted some pre-determined time after displaying a particular stimulus or task via a user interface to patient 100 (e.g., via screen 120). Alternatively, PC 110 may monitor one or more sensors and, when the sensor value passes a pre-determined threshold, PC 110 may initiate transmission of the stimulating signal by device 125. The sensors may include one or more sensors that monitor patient 100 directly (e.g., eye or motion sensors for detecting saccade movement) or user interface sensors (e.g., keyboard button sensors, touch screen sensors, or audible sensors for monitoring speech) to monitor interactions with patient 100. After detecting that the appropriate sensors have been triggered indicating completion of the prompted task by the patient (e.g., by saccade movement of patient 100 or via an appropriate user interface), PC 110 causes device 125 to transmit the appropriate stimulating signal. In some cases, PC 110 will cause the signal to be transmitted as soon as the task is detected as complete. In other implementations, though, PC 110 may include a delay (e.g., 500 msec) before causing the stimulating signal to be transmitted into the patient's brain.

In other implementations, PC 110 monitors a condition of patient 100's brain in order to trigger transmission of the stimulating signal at the optimum time. For example, as described above, the hippocampus is also known to exhibit periodic oscillations in the theta band (e.g., 5-10 Hz) at the time of memory formation. Accordingly, in one particular implementation device 125 may be configured to incorporate a recording electrode disposed within the hippocampus that is placed in communication with PC 110. PC 110 can then monitor the recording electrode in order to detect theta oscillations from that portion of patient 100's brain (in other implementations, other oscillations bands such as alpha, beta, or gamma may be monitored). When those oscillations (or any other predetermined condition) are detected (indicating that memory formation is on-going or imminent), PC 110 can cause device 125 to deliver a stimulating signal in order to assist in memory formation. Again, as described above, in some implementations PC 110 may, rather than deliver the stimulating signal instantly, implement a delay of up to approximately 500 millisecs after detecting theta oscillations before delivering the stimulating signal. In other implementations, the system may monitor the patient's brain to detect oscillations occupying a particular band. Alternatively, the system may monitor for single neural firing, or may be configured to detect particular neurotransmitters such as dopamine, glutamate, or serotonin.

In order to analyze the data received from the recording electrode to detect theta (or other band) oscillations, PC 110 may be configured to perform a Fast Fourier Transform of the raw data received from the recording electrode to identify the frequency components of that data. If oscillatory rhythms are detected in the range of 5-10 Hz (or in some cases, 5-15 Hz), then PC 110 determines that theta oscillations have been detected and takes appropriate steps. In one implementation, PC 110 is configured to require observation of three or more cycles or zero crossings within the theta frequency range before determining that theta oscillations have been detected.

Although in FIG. 3A PC 110 is shown as a separate stand-alone computing device, in some implementations the functionality of PC 110 is incorporated directly into wand 105 or even device 125. In that case, wand 105 or device 125 would be configured to communicate directly (either wired or wirelessly) with the user interface devices and sensors described above.

FIG. 3B is a block diagram illustrating some of the functional components of the present system. Lines between the various components indicate that the components are in communication with one another, where the communication may occur via a wired or a wireless connection.

As shown in FIG. 3B, device 150 is configured to control the delivery of stimulating signals to regions of a brain of a patient in accordance with the present disclosure. Device 150 incorporates microprocessor 152 that is in communication with sensor 154 via analog-to-digital converter 156. Sensor 150 is connected to deep brain electrode 158 for detecting a condition within the patient's brain. For example, deep brain electrode 158, in combination with sensor 154 may be configured to detect oscillations occurring with a brain structure of the patient. Alternatively, sensor 154 may be connected to, or provided by, one or more scalp/cortical EEGs that could alternatively be used to monitor the patient's brain.

Microprocessor 152 is configured to receive digital data from A to D converter 156 describing analog data received from sensor 154. Microprocessor analyzes that data (for example, by detecting theta oscillations, as described above) to determine when to deliver a stimulating signal to the patient's brain. Microprocessor 152 is connected to memory 160, where the software for controlling the operation device 150 may be stored. Device 150 also includes a power source (not shown) for powering microprocessor 152 as well as other components of the device, and for powering the stimulating signal delivered to output 164, as described below.

When microprocessor 152 detects that a condition for stimulating the patient has been met, microprocessor 152 is configured to cause output controller 162 to generate an output stimulating signal that is delivered via output 164. The output is delivered from output 164 to an electrode or other structure or implantable device disposed within the patient's brain for delivering the appropriate stimulation signal 166. In various implementations, the stimulation signal may be delivered by DBS, transcranial magnetic stimulation, optogenetic stimulation, or ultrasonic stimulation.

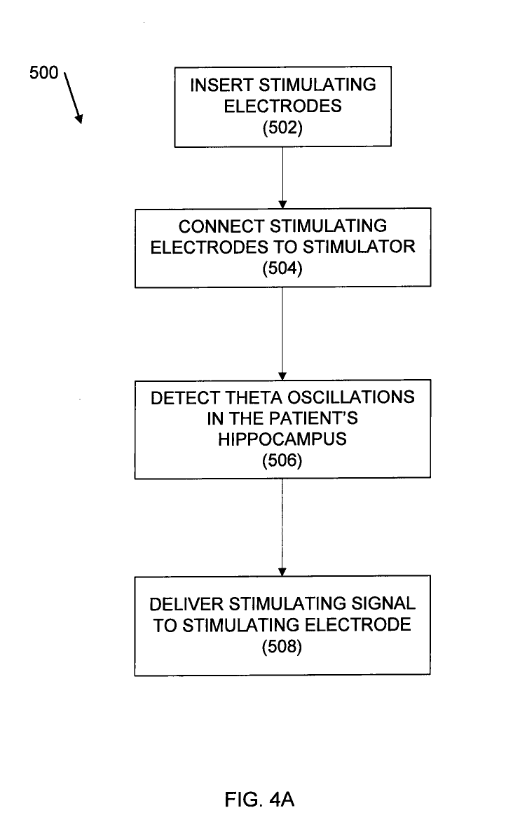

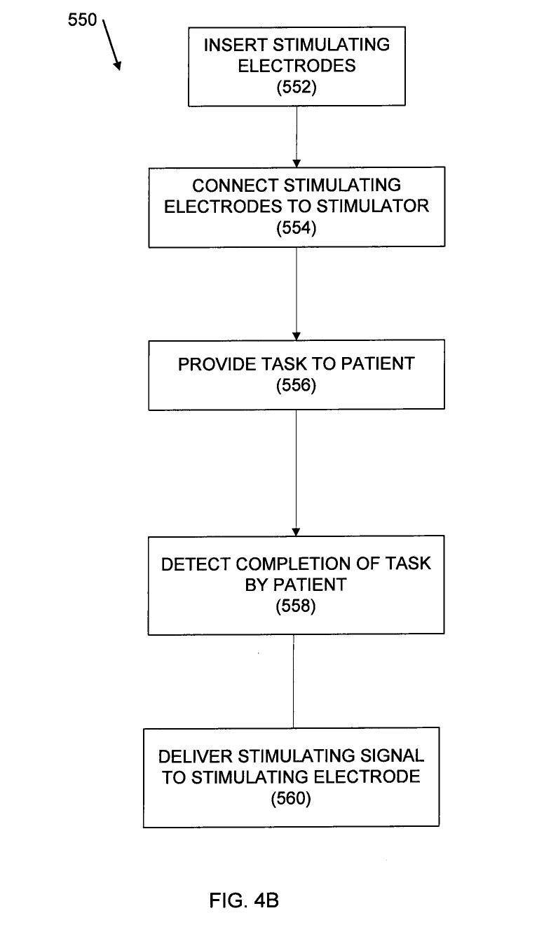

The systems illustrated in FIGS. 3A and 3B are generally configured to control delivery of a stimulating signal to a particular region of a patient's brain. Each system incorporates controllers (or are in communication with controllers) that deliver the stimulating signal at an optimum time for generating therapeutic benefit. FIGS. 4A and 4B are each flowcharts illustrating methods that may be implemented by the controllers of the present system (e.g., PC 110 of FIG. 3A or microprocessor 152 of FIG. 3B) for delivering the appropriate stimulating signal to a patient.

FIG. 4A is a flowchart illustrating method 500 for closed-loop monitoring and delivery of a stimulation signal to a patient that can be used for treating the patient as discussed above. In step 502, stimulating electrodes are inserted into the patient. As discussed above, the stimulation of particular brain structures can be beneficial in stimulating memory formation for the treatment of stroke, dementing disorders, conditions such as depression or motivational problems, brain injuries, or other conditions affecting memory or association formation. For the treatment of dementing disorders, for example, the stimulating electrodes can be inserted into the patient's hippocampus, nucleus basalis of Meynert or mammillary bodies bilaterally. The electrodes may be inserted using any appropriate surgical technique or method including stereotactic techniques.

In step 504 the electrodes are connected to a stimulator that is configured to transmit the stimulating signal through the stimulating electrode into the patient's brain. The combination of the electrode and stimulator may include circuitry for monitoring a condition of the patient (e.g., the presence of theta oscillations, as described above) for controlling delivery of the stimulating signal. Various conditions may be defined, including the presence of oscillations in alpha, beta, gamma, or theta bands in a particular brain structure, single neuronal firing, or the detection of particular neurotransmitters including dopamine, glutamate, or serotonin. Alternatively, the combination may be in communication with an external system (e.g., PC 110 of FIG. 3) that controls delivery of the stimulating signal. In that case, the external system and the electrode/stimulator may communicate with the external system using a wired or wireless connection. Alternatively, the electrode and stimulator combination may be configured to deliver a constant stimulating signal to the brain region in which the stimulating electrode are inserted. In the method illustrated in FIG. 4A, the electrode/stimulator combination is configured to deliver the stimulating signal upon detection of a particular condition in the brain structure of the patient.

The electrode/stimulator combination also includes a battery for powering the unit and (in the case of wireless communications) an RF coil for charging the unit's battery via an external power unit (e.g., wand 105 of FIG. 3A).

In step 506 the condition is detected in the patient's brain. Depending upon the system implementation, the electrode/stimulator combination may analyze data captured from the stimulating electrode (or another recording electrode) to identify the existence of the condition. Alternatively, an external device (e.g., PC 110 of FIG. 3A) may analyze data captured from the stimulating electrode (or a recording electrode, if present) or separate monitoring device to identify the present of theta oscillations. For example, in a system configured to detect theta oscillations, to detect the oscillations, the data retrieved from the electrode may be filtered using a Fast Fourier Transform to detect theta oscillations in the patient's hippocampus occurring within a particular frequency range (e.g., 5-10 or 5-15 Hz, as described above.

After detecting the existence of the condition, in step 508 a stimulating signal is delivered through the stimulating electrode into the patient's brain. In one implementation, the stimulating signal is transmitted through the stimulating electrode for a time duration of 1 second at a frequency of 200 Hz and with a magnitude of 200 micro-amps in 1 microsecond square-wave pulses. Although the stimulating signal may be transmitted as soon as the theta oscillations are detected, in another implementation, the stimulating signal is delivered within 500 msec of the theta oscillations being detected.

As described above, the stimulation of different brain structures can result in improved memory or association formation for the treatment of different disorders. The stimulation may also be used to mitigate the effects of various conditions such as depression or motivational problems. To remedy cognition disorders, for example, the stimulating electrode may be disposed within the patient's Caudate and Nucleus accumbens. Alternatively, to stimulate memory formation, the stimulating electrode may be disposed within the patient's Hippocampus, Nucleus Basalis and/or Mammillary Bodies.

FIG. 4B is a flowchart showing an alternative method 550 for delivering a stimulating signal to a patient for treating memory or brain disorders as discussed above. In step 552, stimulating electrodes are inserted into the patient. As discussed above, the stimulation of particular brain structures can be beneficial in stimulating memory formation for the treatment of various memory or brain disorders, and treating conditions such as stroke, traumatic brain injury, depression or motivational problems. Accordingly, in the present method the stimulating electrodes can be inserted into the patient's Caudate, Nucleus Accumbens, and/or hippocampus bilaterally. The electrodes may be inserted using any appropriate surgical technique or method including stereotactic techniques. In one particular implementation, four stimulating electrodes are inserted into each of the Caudate and Nucleus Accumbens.

In step 554 the electrodes are connected to a stimulator that is configured to transmit the stimulating signal through the stimulating electrode. The combination of the electrode and stimulator may include circuitry for monitoring a condition of the patient for controlling delivery of the stimulating signal. Alternatively, the combination may be in communication with an external system (e.g., PC 110 of FIG. 3) that controls delivery of the stimulating signal to the patient's brain. In that case, the external system and the electrode/stimulator may communicate with the external system using a wired or wireless connection. Alternatively, the electrode and stimulator combination may be configured to deliver a constant stimulating signal to the brain region in which the stimulating electrodes are inserted. In the method illustrated in FIG. 4B, the electrode/stimulator combination is configured to deliver the stimulating signal upon detection of a prompt from an external system (e.g., PC 110 of FIG. 3).

The electrode/stimulator combination also includes a battery for powering the unit and (in the case of wireless communications) an RF coil for charging the unit's battery via an external power/control unit (e.g., wand 105 of FIG. 3A). The electrode/stimulator combination is also configured to receive instructions from the external system via the external power/control unit.

In step 556 the external system (e.g., PC 110 of FIG. 3) prompts the user with a task. The task may include responding to a visual stimulus (e.g., identifying a particular shape on a touch screen, recognizing an individual or an object, or tracking a moving object), responding to an auditory stimulus (e.g., identifying a sound, or identifying a particular tone), or completing a puzzle, or mathematical, or logical problem. The task may be displayed to the patient using any suitable user interface such as computer monitors, touch screens, speakers, projectors, tactile devices, or the like.

In step 558, after prompting the patient with the particular task, the external system detects completion of the task by the patient. The detection may be made via the user interface, for example, or by a sensor system that monitors the patient. For example, if the task requires that the patient type a particular word into a keyboard, the external system can detect completion of the task by monitoring that keyboard. Alternatively, where the patient is tasked with identifying particular shapes on a touch screen, the external system can monitor the touch screen for completion of the task. In some cases, the external system may use a microphone to monitor speech of the patient. If the patient is tasked with speaking a particular sentence, for example, the external system could use a microphone in combination with speech recognition software to detect when the patient has completed the task.

In other implementations, the external system may include movement detectors to identify saccade movements of the patient that may identify completion of the task. The sensors may include motion sensors to detect motion of the patient, or a limb of the patient (e.g., by detecting that the patient has pointed to, or otherwise indicated a particular object as part of a task), or eye sensors, for example, that can detect saccade eye movement of the patient. In those cases, when the external system detects the appropriate movement based upon the task, the external system may consider the task to be complete.

In other implementations, the external system may detect completion of a task by monitoring the patient's brain directly. For example, after prompting the patient with a task, the external system may use implanted electrodes, scalp EEGs, or cortical EEGs to detect brain activity in the patient that is associated with completion of the task.

After the external system detects completion of the task by the patient, in step 560 the external system communicates with the stimulator/electrode combination (e.g., via a wireless communication established between an external wand such as wand 105 of FIG. 3A and an implanted stimulator) to instruct the stimulator to deliver a stimulating signal through the electrode to the region of the brain in which the electrode is implanted. As discussed above, by stimulating the certain brain structures at the time of learning, memory formation, association formation, or other condition treatment can be enhanced. In one implementation, the stimulating signal operates at 200 Hz at an amplitude of 200 micro-amps with 1 microsec square-wave pulses and is triggered for a duration of 1 sec. Although the stimulating signal may be transmitted immediately upon detecting completion of the task at hand, in one implementation the stimulating signal is delivered within 500 millisec of task completion detection.

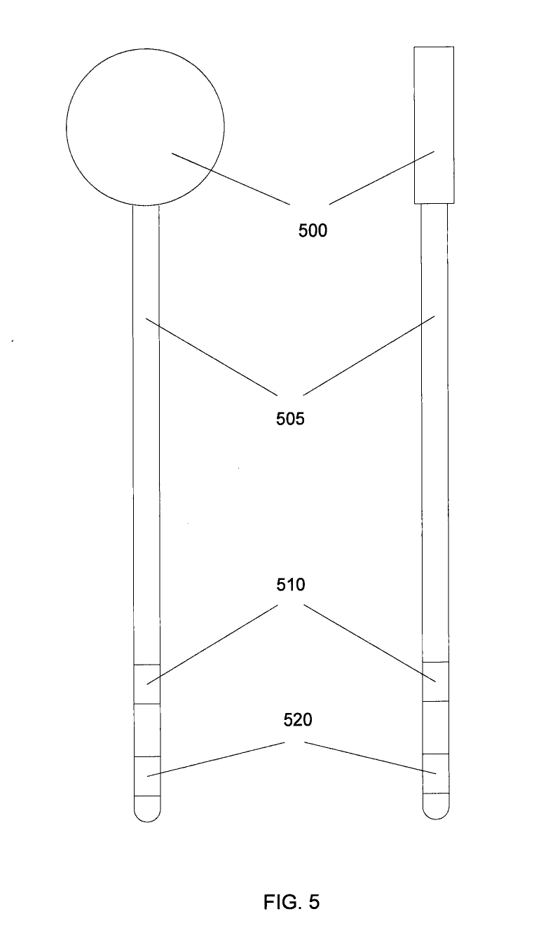

FIG. 5 illustrates functional components of an implantable device that may be used in accordance with the present disclosure (see, for example, element 125 of FIG. 3). The device includes two electrodes that can, together, operate as current delivery and current return electrodes. A distal end of the devices includes electrodes 510 and 520 that are used to deliver stimulating signals to or return stimulating signals from various regions of the brain. The electrodes 510 and 520 can be constructed using platinum, platinum iridium, titanium, tantalum, iridium oxide, titanium nitride, conductive polymer, tungsten or other biocompatible materials. The electrodes 510 and 520 are connected by conductors in insulated, elongated portions 505 of the device body to circuit portions 500 of the implanted device. While two electrodes 510 and 520 are shown, it may be advantageous to have just one with the current return electrode located elsewhere; or it may be advantageous to use many more electrodes. The insulation that protects the conductors and circuit from the implant environment may be silicone, polyurethane, a block co-polymer or other suitable biocompatible material. Alternatively the circuit portion 500 may be packaged in a hermetic can with feedthroughs.

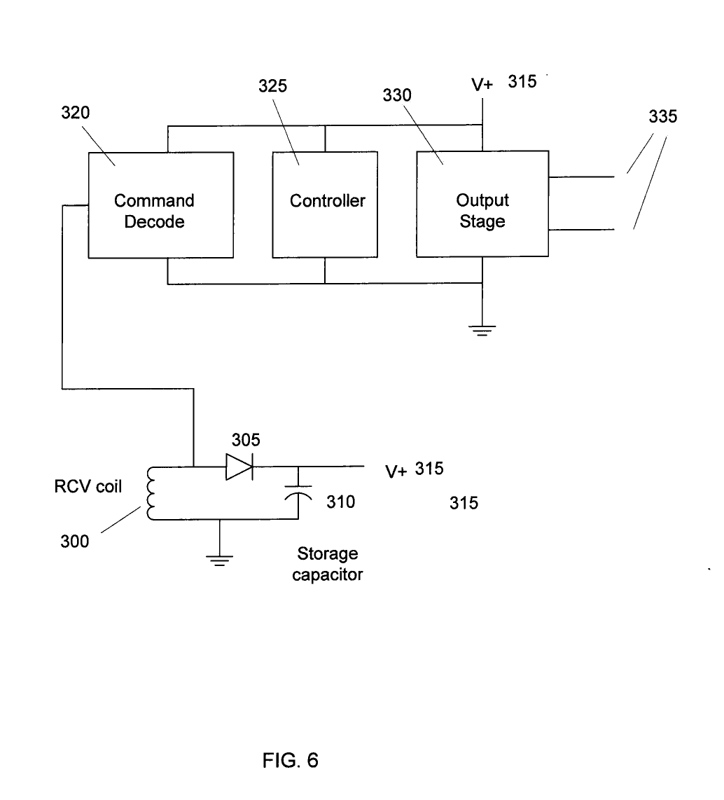

FIG. 6 is a block diagram illustrating functional components of the present implantable device (e.g., device 125 of FIG. 3A). Coil 300 is used to receive power and commands from an external wand (not shown, see, for example, wand 105 of FIG. 3). The power is transmitted to coil 300 using electromagnetic coupling. In one implementation, the power transmission occurs using a power signal operating at an alternating voltage at approximately 200 kHz. The power transmission is conducted through diode 305 to storage capacitor 310, resulting in a voltage supply being generated across the capacitor (e.g., V+ 315). V+ 315 is typically in the range of 2 to 10 volts and is used to power the other circuit blocks, however, depending upon the system implementation the voltage V+ can be adjusted.

Commands transmitted through the wand by an external control system (e.g., PC 110 of FIG. 3A) can be encoded within the power signal, for example, as phase shift keying or as gaps in the power signal. The command decode block 320 contains an oscillator or other time base circuitry and decodes the coded commands and confirms that there are no communication errors, for example, by parity bit checking, CRC or other data integrity check. Coded commands may include the amplitude, pulse width, number and timing of stimulation pulses to be applied to the patient. The decoded command is presented to a controller block 325 that translates the command code into logic for controlling the output stage 330. The output stage 330 is connected to the patient by conductors 335 that connect to electrodes (e.g., conductors 510 and 520 of FIG. 5) which are in contact with the patient's brain. The output stage includes a voltage reference and electronic switches that allows the output stage to convert V+ 315 into stimulation pulses conducted to the patient along conductors 335.

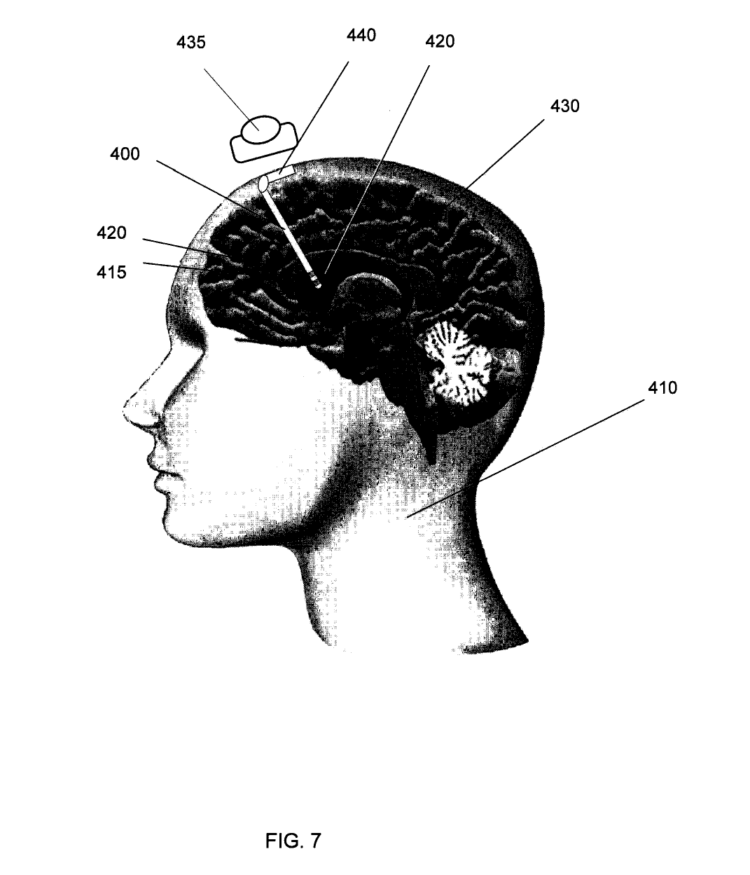

FIG. 7 is an illustration showing the present device 400 implanted in the patient's head 410. The device electrodes 415 and 420 are shown implanted in the anterior portion of the caudate 425 (a large structure in the brain 430). An external wand 435 (see, for example, wand 105 of FIG. 3) contains a coil for communicating power and commands to the implanted coil 440. The decoded commands result in electrical stimulation being applied through the electrodes 415 and 420 to the patient's caudate 425.

While the invention is described through the above-described exemplary embodiments, it will be understood by those of ordinary skill in the art that modifications to, and variations of, the illustrated embodiments may be made without departing from the inventive concepts disclosed herein. Moreover, while the preferred embodiments are described in connection with various illustrative data structures, one skilled in the art will recognize that the system may be embodied using a variety of data structures. Furthermore, disclosed aspects, or portions of these aspects, may be combined in ways not specifically listed above. Accordingly, it is felt therefore that the scope of protection provided by this patent should not be viewed as limited by the above description, but rather should only be limited by the scope of the below claims.

1. A method for stimulating a brain structure of a patient for treating a condition, comprising:
inserting a stimulating electrode into the brain structure of the patient;
prompting the patient with a task; and
when the task is completed by the patient, using the stimulating electrode to transmit a stimulating signal into the brain structure of the patient.

2. The method of claim 1, including using a sensor to detect completion of the task by the patient.

3. The method of claim 2, wherein the sensor is configured to detect a movement of the patient.

4. The method of claim 3, wherein the movement is a saccade movement.

5. The method of claim 1, wherein the stimulating signal has a duration of approximately 1 second, a frequency of approximately 200 Hertz and a magnitude of approximately 200 microamps.

6. The method of claim 1, wherein the stimulating signal comprises an approximately 0.1 millisecond per phase square wave.

7. The method of claim 1, wherein the stimulating electrode is disposed within at least one of a Hippocampus, Nucleus Basalis of Meynert, Mammillary Body, Caudate and Nucleus accumbens of the patient.

8. The method of claim 1, wherein the stimulating signal is into the brain structure of the patient within approximately 500 milliseconds of detecting completion of the task by the patient.

9. A method for stimulating a brain structure of a patient for treating a condition, comprising:
inserting a stimulating electrode into the brain structure of the patient;
monitoring the brain of the patient for a condition; and
when the condition is detected in the brain of the patient, using the stimulating electrode to transmit a stimulating signal into the brain structure of the patient.

10. The method of claim 9, wherein the condition includes at least one of an existence of at least one of alpha, beta, gamma, and theta oscillations within the brain structure, a firing of a single neuron, and an existence of a neurotransmitter including at least one of dopamine, glutamate, and serotonin.

11. The method of claim 9, wherein monitoring the brain of the patient includes using a fast Fourier transform to analyze electrical activity in the brain of the patient.

12. The method of claim 9, wherein the stimulating signal has a duration of approximately 1 second, a frequency of approximately 200 Hertz and a magnitude of approximately 200 microamps.

13. The method of claim 9, wherein the stimulating signal comprises an approximately 0.1 millisecond per phase square wave.

14. The method of claim 9, wherein the stimulating electrode is disposed within at least one of a Hippocampus, Nucleus Basalis of Meynert, Mammillary Body, Caudate and Nucleus accumbens of the patient.

15. The method of claim 9, wherein the stimulating signal is transmitted into the brain structure of the patient within approximately 500 milliseconds of detecting the theta oscillations.

16. A system for stimulating a brain structure of a patient for treating a condition, comprising:
a stimulating electrode configured to be disposed into a brain structure of the patient; and
a controller configured to:
prompt the patient with a task; and
when the task is completed by the patient, use the stimulating electrode to transmit a stimulating signal into the brain structure of the patient.

17. The system of claim 16, including using a sensor to detect completion of the task by the patient.

18. The system of claim 17, wherein the sensor is configured to detect a movement of the patient.

19. The system of claim 18, wherein the movement is a saccade movement.

20. The system of claim 16, wherein the stimulating signal has a duration of approximately 1 second, a frequency of approximately 200 Hertz and a magnitude of approximately 200 microamps.
